(19) United States
(12) Patent Application Publication
(10) Pub. No.: US 2008/0011053 A1
(43) Pub. Date: Jan. 17, 2008

(54) Soot sensor and operating method

(75) Inventors: Maximilian Fleischer, Hohenkirchen (DE); Roland Pohle, Herdweg (DE); Kerstin Wiesner, Putzbrunn (DE)

Correspondence Address: COHEN, PONTANI, LIEBERMAN & PAVANE, 551 FIFTH AVENUE, SUITE 1210, NEW YORK, NY, 10176, US

(73) Assignee: Siemens Aktiengesellschaft

(21) Appl. No.: 11/827,029

(22) Filed: Jul. 10, 2007

(30) Foreign Application Priority Data
Jul. 11, 2006 (DE) 10 2006 032 106.5

(51) Int. Cl. G01N 33/00; G01N 29/02

(52) U.S. Cl. 73/23.31; 73/24.03

(57) ABSTRACT

A soot sensor includes a plurality of sensor elements including a base body having at least a part that is excitable to produce mechanical oscillations, the base body having at least one defined surface having predefined catalytic properties and subjected to a measurement gas, and a heating element acting on said base body, wherein a change in an oscillation frequency, an oscillation amplitude or the quality of the oscillation which has occurred due to increasing precipitation of soot on the defined surface indicates the presence of soot.

### BACKGROUND OF THE INVENTION

1. Field of the Invention

The invention relates to a sensor for detecting soot and a method for operating this sensor to reliably detect soot which has precipitated out from a specific volume of gas.

2. Description of the Related Art

The increase in carbon dioxide in the atmosphere and the associated costs or effects of this increase on the environment and humans have recently become a constant topic of discussion. In addition, fossil fuels are available in only a finite supply but combustion processes are used to a wide extent in order to obtain energy. As such, continuous developments are being pursued to optimize the thermodynamic efficiency of these combustion processes. Other phenomena related to the above issues are also developing. For example, in the field of motor vehicles there is an increase in the use of diesel vehicles. The disadvantage of diesel combustion technology is that it produces significantly increased soot emissions compared to optimized spark ignition engines. Furthermore, these soot emissions are virtually impossible to prevent through combustion measures. Moreover, the soot is highly carcinogenic, particularly due to the deposition of polycyclic aromatic compounds (PAK). Legislators have reacted to these developments and imposed corresponding exhaust gas emission standards which are made more stringent on a regular basis. For example, maximum limits for soot emissions are prescribed. The above developments and legislation have prompted a further development of sensor systems for reliably measuring soot content in exhaust gases.

The application of soot sensors can be categorized in various ways. For example, soot sensors may be distinguished according to the measures which are respectively triggered by the presence of sensed quantities of soot.

On the one hand a soot sensor can measure the amount of soot emitted at a particular time and thus provide information to an engine management system in the current driving situation of a motor vehicle to reduce the emissions with adaptations using control technology.

On the other hand, active exhaust gas cleaning is carried out by means of what are referred to as exhaust gas soot filters. These are filters which can be regenerated and which filter significant parts of the soot content out of the exhaust gas. In this context, soot sensors are required to monitor the functioning of the soot filters or to control their regeneration cycles.

Furthermore, soot sensors with sufficient measuring accuracy are also to be used to measure the soot content in the air in the vicinity of roads.

The soot sensors may also be categorized by how they detect soot. There are various approaches to detecting soot. One approach which has been pursued by using laboratory equipment is to employ light scattering by the soot particles. This procedure is suitable for costly laboratory measuring equipment. Attempts to use this technology as a mobile sensor in exhaust gas have failed to produce a cost-effective sensor in motor vehicles. The design of optical elements entails high costs, and the problems of the soiling of, for example, optical windows through emissions from combustion are difficult to solve.

Another technology for detecting soot which can be put into practice is described by a thermal method. In this case, the sensor is composed of an open pore shaped body, for example a honeycomb-shaped ceramic body, a heating element and a temperature sensor. Soot is deposited on the body. For the measurement, the soot which is deposited in a prescribed time period is ignited using the heating element and burnt off. The increase in temperature arising during the combustion is measured and correspondingly used as an indication of the amount of soot deposited. Even though this is certainly a practical procedure under constant ambient conditions, the measurement of the relatively small increase in temperature proves a difficult problem to resolve under the conditions in a motor vehicle exhaust gas section with highly fluctuating flows and exhaust gas temperatures.

Electrical methods for measuring soot can be based on two different principles. According to one method, gas to which soot is applied is located in an electrical field between two electrodes. An exhaust gas stream which is charged with soot is used to produce an ionization current. One embodiment of this principle is described, for example, in DE 102 44 702. The exhaust gas stream passes the two electrodes which are provided with an electrical insulation layer and between which the soot-containing gas to be examined is located. The electrodes are operated with an alternating voltage between 1 kV and 10 kV, with a dielectrically impeded discharge occurring between the electrodes as a function of the concentration of soot. The occurring currents are measured. This procedure can be reliably applied in a motor vehicle. However, the implementation requires high voltages and complex measuring technology. For this reason, cost-effective implementation of this particular method is some way off. Furthermore, the alternating gaseous exhaust gas components cause significant falsification of the measurements by influencing the ionization current.

Novel sensors for detecting soot which use the electrical conductivity of the soot are described, for example, in the German patent application with the file number 10 2005 030 134.7. As the deposition on the base body increases, the conductivity of an insulating base body on which two electrodes are mounted will also increase. A sensor of this type has, for example, the particular advantage of self-monitoring.

### SUMMARY OF THE INVENTION

An object of the present invention is to provide a sensor for the precise measurement of quantities of soot with a simple structural design. Another object of the present invention is to provide an operating method is intended to ensure precise measurement of the soot without significant interference variables.

The object is met by a soot sensor, comprising a plurality of sensor elements including a base body having at least a part that is excitable to produce mechanical oscillations, the base body having at least one defined surface with predefined catalytic properties and subjected to a measurement gas. The sensor elements further include a heating element acting on the base body, wherein a change in an oscillation frequency, an oscillation amplitude or the quality of the oscillation which has occurred due to increasing precipitation of soot on the defined surface is an indication of the presence of soot.

The object is also met by a method for operating the soot sensor including the steps of heating, in a measuring phase, the base body to a predefined first temperature higher than 100° C. so that only soot is deposited on the base body, determining a mass of the precipitated soot by measuring a change in the oscillation frequency, and heating, in a regeneration phase, the base body to a predefined second temperature when a maximum mass of precipitated soot is measured in the measuring phase so that the precipitated soot is burnt with residual oxygen.

The deposition of soot on a sensor element naturally changes the mass of the sensor element. The change in mass which is caused by the deposition of soot is used as a measurement variable in the procedure described here. The reading-out of this measurement variable is carried out by measuring the change in an oscillation frequency of the sensor element, which may be in particular a resonant frequency.

A soot measurement can easily be carried out by using a sensor essentially composed of a base body which can be made to oscillate. The base body is made to oscillate mechanically or is excited to oscillate mechanically entirely or partially by electrical excitation. This excitation can occur through different physical effects such as the piezo-mechanical effects of capacitive transducers. The base body has at least one surface which is subjected to soot-containing gas and which has defined properties for catalytically burning precipitated soot. The sensor for measuring within a measurement phase is heated to a first predetermined temperature and kept there by a heating element mounted on the base body. If soot from the soot-containing exhaust gas is precipitated on the surface, the precipitated soot brings about a change in the frequency of the sensor element. This change in the oscillation frequency can serve as a measurement variable for the presence of the film of soot.

The sensor element is heated to a constant first temperature during the measurement phase and held there, the temperature being above 100° C. At this first temperature, it is intended to prevent undesired exhaust gas components such as, in particular, moisture, NOx or SO₂ which could bring about a change in mass on the surface of the sensor element or base body which influences the signal, from being deposited. The deposition of soot particles does, however, take place at this temperature. As the deposition of soot particles increases, there is finally a change in the oscillation frequency which is correlated directly to the mass of the precipitated film of soot. During this phase of the collection of soot particles in the measurement phase, the temporal change in the frequency serves as a measure of the average loading of the gas with soot particles. When a specific change in frequency is exceeded, the sensor element is heated to a defined second temperature which is higher than the first temperature. In such a case, the soot particles are burnt off with the residual oxygen present in the exhaust gas, which constitutes the regeneration phase. Subsequent to the regeneration phase, it is possible, depending on the predefinition of the controller, to initiate the next measuring phase, at the start of which measuring phase the natural frequency of the oscillating body can be newly determined.

In one embodiment, at least two sensor elements are used, in which case at least one of the sensor elements is always in the measurement phase, and thus a continuous, substantially interruption-free measurement is ensured.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the text which follows, exemplary embodiments are described with reference to accompanying schematic figures which do not restrict the invention, the following being illustrated in particular:

FIGS. 1a and 1b are side and plan views of a soot sensor according to the invention with piezo-electric excitation;

FIG. 2 is a schematic plan view of a heating structure which can be used as a temperature sensor if a suitable conductor track material is used,

FIG. 3 is a schematic plan view of interdigital electrodes for detecting soot by measuring conductivity, and

FIG. 4 is a schematic sectional view of a structure having capacitive excitation of oscillations; and

FIG. 5 is a schematic sectional view of the structure of FIG. 4 in a different state.

### DETAILED DESCRIPTION OF THE PRESENTLY PREFERRED EMBODIMENTS

According to an embodiment of the present invention, a part of a sensor that is electrically excitable to oscillate is composed at least partially of a piezo-electric material. Examples of temperature stable piezo-electric materials which can be excited to undergo volume oscillations at temperatures of at least 900° C. include quartz, gallium orthophosphate or langasite. Other materials with a high Curie temperature such as for example, gallium phosphate GaPO₄ and lithium niobate LiNbO₃ are also suitable. According to another embodiment, a second temperature-resistant material may be used in which the hot surface on which soot is deposited is thermally insulated from the piezo-electric material so that a cost-effective conventional piezo-electric material such as for example, lead zirconate titanate (PZT) piezo-ceramic, zinc oxide or organic materials such as polyvinylidene difluoride (PVDF) can be used.

The mechanical oscillations may alternatively be excited electrostatically.

An excitation voltage is applied to the piezo-electric material by corresponding electrodes. If soot is deposited, an additional undesired current path may arise between these electrodes. For this reason, the surface which is subjected to the measuring gas is covered with a layer which is a good electrical insulator. As a result, very good electrical insulation of the electrodes is achieved and an undesired influence of the deposition of soot on the excitation is avoided. A ceramic layer which is a very good electrical insulator, for example, highly pure aluminum oxide Al₂O₃ or AlN, is suitable as a material for the insulation layer. Likewise, highly insulating layers of SiO₂ or SiN which are applied by means of a suitable layer precipitation method such as sputtering or CVD can be used.

To support the catalytic burning off of the soot, the catalytic activity of the surface is selectively influenced to oxidize soot deposited on the surface to form volatile gas components. This is done by applying an oxidation catalyst to the surface, this in the form of a dispersion so that individual regions which are not coherent are produced and no undesired conductivity is established by this additional layer. Materials for such catalysts are, for example, platinum metals such as Pt, Ra, Pd or their alloys. Catalytically active oxides can also be used, these being oxides of transition group metals such as Fe₂O₃, CeO₂, Mn₂, Cr₂O₃, HfO₂.

The heating element is composed of a metallic conductor track, for example, made of platinum or a platinum metal or its alloys. The heating element used here is associated with a specific resistance which constitutes a function of the temperature of the sensor element so that the temperature can be determined by evaluating the current resistance of the heating element. In this case, it is possible to dispense with a separate temperature sensor in the sensor element.

For the operating method of the sensor, the precise knowledge of the temperature is necessary, regardless of how it is determined. To protect the heating element and the temperature sensor against aging due to environmental influences, heating element is protected against contact with the environment. This is done by applying it to a surface of the base body and providing it with an additional cover layer. Materials for this are glasses with a high melting temperature, aluminum oxide or silicon dioxide or a combination thereof. The excitation electrodes are composed, for example of metals which are stable in exhaust gas, such as Pt, Rh, alloys of the platinum metals or chromium alloys and nickel alloys, in which case further electrically conductive compounds which are stable in exhaust gas can be formed by TiN, BN, SiC, BC, PtSi.

By additionally applying electrodes which are stable in exhaust gas for measuring conductivity, it is possible to integrate this method of detecting soot with the described sensor element so that, in fact, the mass of soot can be recorded simultaneously by means of the change in the resonant frequency and by means of the change in the conductivity independently of two measurement variables.

In terms of the type of the oscillating body, it can be a volume thickness oscillator, a volume shear-type oscillator, a love-wave type oscillator, a surface wave component, oscillating diaphragms such as, for example, capacitive micromechanical ultrasonic transducers or cantilever oscillators.

Advantages of the invention are a compact, simple and thus cost-effective design with corresponding operating methods is specified for determining the soot content in exhaust gases. The design is constructed from materials which give it the required resilience and resistance to aggressive and corrosive environmental conditions, for example, even in the exhaust gas. The sensor is suitable for continuously monitoring the exhaust gases and requires no maintenance or replacement or consumables at all.

As a result of the metering method with a cyclical operating mode, the measuring principle makes direct reference to the regulations of the exhaust gas standard which regulates soot emissions per 100 km traveled. The displacement of the oscillating frequency means that the mass of precipitated soot can be specified absolutely, thus supplying quantitative information.

Combining the frequency measurement and the measurement of conductivity provides information about the quantity and the properties of the soot particles such as, for example, the particle sizes.

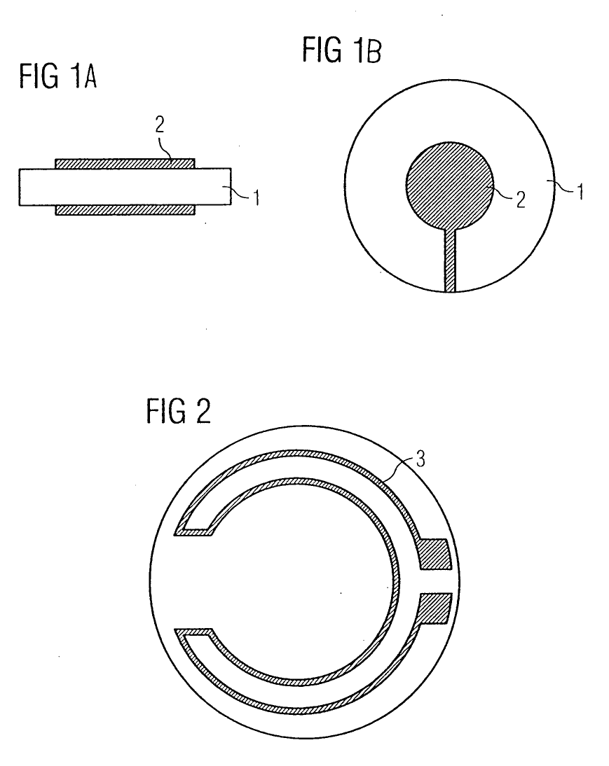

The basic design of a soot sensor according to an embodiment of the invention with piezo-electric excitation is shown schematically in FIGS. 1A and 1B. A base body 1 of the sensor composed of piezo-electric material is of circular design to minimize the occurrence of secondary modes when oscillations are excited. The excitation is carried out by two electrodes 2 arranged on each side and which are circular in this case. On the rear of the sensor an annular heating structure is located, and said structure can serve at the same time as a temperature sensor.

FIG. 2 is a schematic illustration of the heating element 3 of the annular heating structure which can be divided basically into connecting pads 3a and conductor tracks 3b. As a result of the dependence of the resistance of the heating element on the heating temperature, the heating element can simultaneously be used as a temperature sensor.

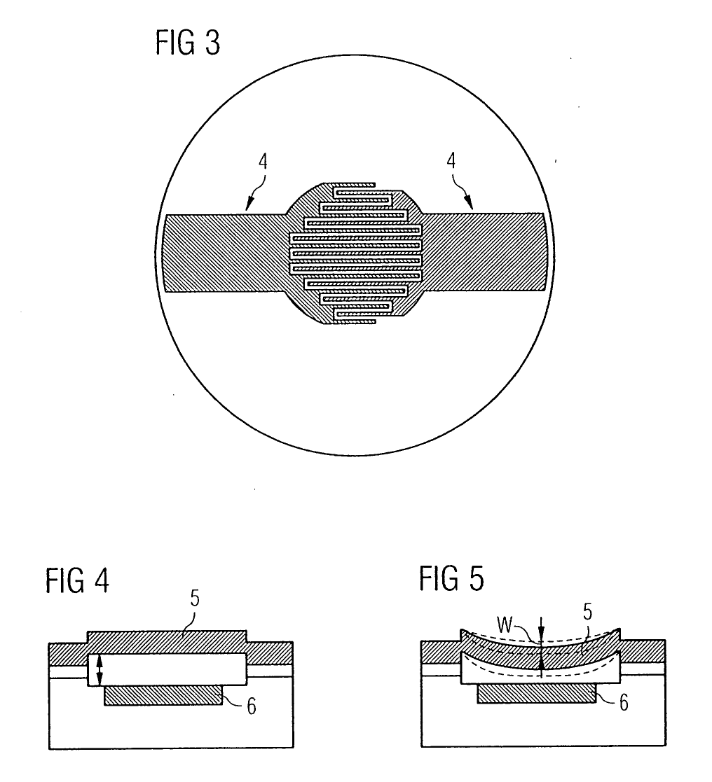

According to FIG. 3, the upper side of a sensor according to another embodiment is provided with linear interdigital electrodes 4 between the broad contact faces. This structure improves the detection properties since, compared to a structure without finger electrodes, a conductive path is produced, even when there is a slight covering of soot. The evaluation of the change in conductivity according to FIG. 3 can additionally be used to change the oscillation properties of an oscillating element so that more wide-ranging evaluations are possible. As an alternative to the piezo-electric excitation, a diaphragm may be capacitively deflected on a periodic basis by a corresponding electrode.

FIG. 4 shows a corresponding design in the state of rest and FIG. 5 shows a diaphragm with corresponding deflection W of a diaphragm 5, which is caused by the corresponding electrode 6. For all excitation variants, a design as a diaphragm which is suspended in a carrier substrate is also possible.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A soot sensor, comprising a plurality of sensor elements including:
a base body having at least a part that is excitable to produce mechanical oscillations, said base body having at least one defined surface having predefined catalytic properties and subjected to a measurement gas; and
a heating element acting on said base body,
wherein a change in an oscillation frequency, an oscillation amplitude or the quality of the oscillation which has occurred due to increasing precipitation of soot on the defined surface is an indication of the presence of soot.

2. The soot sensor of claim 1, wherein the oscillation frequency is a resonant frequency of said sensor element.

3. The soot sensor of claim 1, wherein said at least one part of said base body comprises piezo-electric material.

4. The soot sensor of claim 3, wherein said at least one part of said base body further comprises temperature-resistant, insulating material providing thermal insulation of the piezo-electric material with respect to said at least one defined surface.

5. The soot sensor of claim 1, wherein the mechanical oscillations are applied electrostatically.

6. The soot sensor of claim 1, further comprising an electrically insulating layer protecting the elements of said soot sensor subjected to the measurement gas.

7. The soot sensor of claim 1, further comprising a layer of an oxidation catalytic converter as dispersion covering the elements on which soot can precipitate.

8. The soot sensor of claim 1, further comprising a temperature measuring element.

9. The soot sensor of claim 1, wherein said heating element is composed of a metallic conductor track which simultaneously functions as a temperature sensor.

10. The soot sensor of claim 8, further comprising an anti-corrosive layer covering said heating element and said temperature measuring element.

11. The soot sensor of claim 1, further comprising electrodes for exciting the mechanical oscillations, said electrodes being composed of a metal which is stable in exhaust gas.

12. The soot sensor of claim 11, wherein a change in conductivity due to the precipitation of soot between electrodes further indicates the presence of soot.

13. A method of operating a soot sensor, wherein the soot sensor comprises a plurality of sensor elements including a base body having at least a part that is excitable to produce mechanical oscillations, the base body having at least one defined surface having predefined catalytic properties and subjected to a measurement gas, and a heating element acting on said base body, wherein a change in an oscillation frequency, an oscillation amplitude or the quality of the oscillation which has occurred due to increasing precipitation of soot on the defined surface indicates the presence of soot, the method comprising the steps of:
heating, in a measuring phase, the base body to a predefined, first temperature higher than 100° C. so that only soot is deposited on the base body;
determining a mass of the precipitated soot by measuring a change in the oscillation frequency; and
heating, in a regeneration phase, the base body to a predefined second temperature when a maximum mass of precipitated soot is measured in the measuring phase so that the precipitated soot is burnt with residual oxygen.

14. The operating method of claim 13, maintaining a long-term measuring cycle by continuously repeating the measuring and regeneration phases.

15. The operating method of claim 13, performing an uninterrupted measurement of the soot content using at least two of the soot sensors, such that at least one of the at least two sensors in the measuring phase.

16. The operating method of claim 13, wherein the predefined second temperature is between 600 and 900° C.
